(19) United States
(12) Patent Application Publication
(10) Pub. No.: US 2022/0280086 A1
(43) Pub. Date: Sep. 8, 2022

(54) MANAGEMENT SERVER, METHOD OF GENERATING RELATIVE PATTERN INFORMATION BETWEEN PIECES OF IMITATION DRAWING DATA, AND COMPUTER PROGRAM

(71) Applicant: RFCAMP LTD., Gyeonggi-do (KR)

(72) Inventors: Jae Hyung RYU, Gyeonggi-do (KR); Kwon Soo KIM, Seoul (KR); Ji Youn KIM, Gyeonggi-do (KR)

(73) Assignee: RFCAMP LTD., Gyeonggi-do (KR)

(21) Appl. No.: 17/681,205

(22) Filed: Feb. 25, 2022

(30) Foreign Application Priority Data
Mar. 3, 2021 (KR) 10-2021-0028355

(51) Int. Cl. A61B 5/16; G06V 10/74; G06V 10/56

(52) U.S. Cl. CPC A61B 5/165; G06V 10/761; G06V 10/56

(57) ABSTRACT

The disclosure provides a method of generating relative pattern information between imitation drawing data. By generating pattern information obtained by digitally analyzing one or more pieces of drawing data of an imitation data group, the generated pattern information may be used to analyze psychological state values of users in the imitation data group. Also, a connection between the psychological state values of the users in the imitation group may be discriminated by generating relative pattern information between N pieces of drawing data, which are similar in shape and color. In addition, the relative pattern information may be analyzed by using a model trained by machine-training.

### CROSS-REFERENCE TO RELATED APPLICATION

This application is based on and claims priority under 35 U.S.C. § 119 to Korean Patent Application No. 10-2021-0028355, filed on Mar. 3, 2021, in the Korean Intellectual Property Office, the disclosure of which is incorporated by reference herein in its entirety.

### BACKGROUND

### 1. Field

One or more embodiments relate to a management server, a method of generating relative pattern information between imitation drawing data, and a computer program.

### 2. Description of the Related Art

In the existing art therapy, drawing techniques are used very exceptionally. Most of the existing art therapy techniques combine counseling and drawing, and a method of analyzing the psychology of a counselee by analyzing the shape and color of a drawing drawn by the counselee according to a topic suggested by an expert of art therapy has been mainstream. However, in this existing art therapy technique, a dialogue to form a bond between a counselee and an expert of art therapy and revealing of extremely personal family history and worries are needed to be performed first. The expert may suggest a topic for drawing by considering the age or maturity level of the counselee and provide a drawing material suitable for the topic for drawing to allow the counselee to draw. In the operation that the counselee draws, the expert intervenes with the drawing operation of the counselee and analyzes the psychology of the counselee based on a completed drawing. In offline art psychotherapy, a drawing drawn by the counselee may be analyzed through visual parts such as shape and color.

However, in the case of acquiring a drawing through a digital device, drawings having similar visual parts such as shape and color may have different digital patterns.

Offline art psychotherapists analyze digital drawings as information that may be seen with the naked eye, and may not analyze the digital drawings by using digital pattern information.

### SUMMARY

One or more embodiments include a management server, a method, and a computer program, which may be used to analyze psychological state values of users in an imitation data group by generating pattern information obtained by digitally analyzing one or more pieces of drawing data of the imitation data group.

Additional aspects will be set forth in part in the description which follows and, in part, will be apparent from the description, or may be learned by practice of the presented embodiments of the disclosure.

According to one or more embodiments, a method of generating relative pattern information between imitation drawing data includes collecting a first piece of drawing data generated by a first user and a second piece of drawing data generated by a second user and imitated from the first piece of drawing data, generating a first imitation data group including the first piece of drawing data and the second piece of drawing data, obtaining, from the first imitation data group, a first piece of pattern information and a second piece of pattern information respectively corresponding to the first piece of drawing data and the second piece of drawing data, and calculating a first psychological state value and a second psychological state value respectively corresponding the first piece of pattern information and the second piece of pattern information, determining a relative psychological state value of the second psychological state value with respect to the first psychological state value, and calculating relative pattern information of the second piece of pattern information with respect to the first piece of pattern information, and generating the relative psychological state value and the relative pattern information generated in the first imitation data group as a first imitation emotion pattern related to the first imitation data group.

The method of generating relative pattern information between imitation drawing data according to embodiments of the present disclosure may further include receiving a third piece of drawing data generated by a third user, and, in response to the third piece of drawing data having a degree of similarity with the first or second piece of drawing data, the degree of similarity being greater than or equal to a preset minimum similarity value, generating a third piece of pattern information corresponding to the third piece of drawing data by using the first imitation emotion pattern, and determining a third psychological state value corresponding to the third piece of pattern information.

The method of generating relative pattern information between imitation drawing data according to embodiments of the present disclosure may further include determining one of the first and second users as an original author and the other one as an imitator by using the relative psychological state value and the relative pattern information.

The method of generating relative pattern information between imitation drawing data according to embodiments of the present disclosure may further include receiving a third piece of drawing data generated by a third user, and, in response to the third piece of drawing data having a degree of similarity with the first or second piece of drawing data, the degree of similarity being greater than or equal to a preset minimum similarity value, calculating a third piece of pattern information corresponding to the third piece of drawing data, and managing relative pattern information of the third piece of pattern information with respect to the first piece of pattern information by including the relative pattern information of the third piece of pattern information in the first imitation emotion pattern.

The method of generating relative pattern information between imitation drawing data according to embodiments of the present disclosure may further include generating an imitation order map based on a generation time between the first piece of drawing data and the second piece of drawing data, the first piece of drawing data and the second piece of drawing data of the first imitation data group.

The method of generating relative pattern information between imitation drawing data according to embodiments of the present disclosure may further include creating a community of one or more users who have drawn according to the first piece of drawing data as a group, and sharing drawing data of the one or more users in an online area of the community.

The first and second pieces of drawing data may each include at least one of a time value, a number of strokes, a stop time value, an erase time value, start coordinates, last coordinates, pen pressure, a velocity, a thickness, a color value, a red, blue, and green (RGB) ratio, a brightness value ratio, a hue ratio, and a color ratio.

According to one or more embodiments, a computer program is stored in a computer-readable storage medium configured to execute one of methods according to embodiments of the present disclosure.

In addition, another method for implementing the present disclosure, another system, and a computer-readable recording medium for recording a computer program configured to execute the method are further provided.

Other aspects, features and advantages other than those described above will become apparent from the following drawings, claims, and detailed description of the present disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a block diagram of an electronic device according to embodiments of the present disclosure;

FIG. 2 is a block diagram of a psychological state discrimination unit according to embodiments of the present disclosure;

FIG. 3 is a block diagram of a drawing data analysis unit according to embodiments of the present disclosure;

FIG. 4 is a diagram of a psychology discrimination system according to embodiments of the present disclosure;

FIG. 5 is a block diagram of a database and a management server;

FIG. 6 is a flowchart of a method of generating relative pattern information between imitation drawing data, according to embodiments of the present disclosure;

FIG. 7 shows an example of a correlation table showing pattern information used for psychological state discrimination and each type of psychological state value;

FIG. 8 shows an example of an imitation order of first to eleventh pieces of drawing data of an imitation data group;

FIG. 9 shows an example illustrating a relative relationship between data values of first to eleventh pieces of drawing data of an imitation data group; and

FIG. 10 shows an example of psychological state values from first to eleventh pieces of drawing data of an imitation data group.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, the configuration and operation of the present disclosure are described in detail with reference to embodiments of the present disclosure shown in the accompanying drawings.

As the present disclosure allows for various changes and numerous embodiments, particular embodiments will be illustrated in the drawings and described in detail in the written description. Effects and features of the present disclosure, and a method of achieving them will become apparent with reference to embodiments described below in detail in conjunction with the drawings. The present disclosure may, however, be embodied in many different forms and should not be construed as being limited to the example embodiments set forth herein.

Hereinafter, the present disclosure will be described in detail by explaining embodiments of the present disclosure with reference to the attached drawings. When describing with reference to the drawings, like reference numerals in the drawings denote like or corresponding components, and redundant descriptions thereof are omitted.

In the present specification, terms such as “training” and “learning” are not intended to refer to psychological actions such as human educational activities, but are interpreted as terms referring to performing machine learning through computing according to a procedure.

In the following embodiments, while such terms as “first,” “second,” etc., are used to distinguish one component from another instead for limitation.

In the following embodiments, an expression used in the singular encompasses the expression of the plural, unless it has a clearly different meaning in the context.

In the following embodiments, it is to be understood that the terms such as “including,” “having,” and “comprising” are intended to indicate the existence of the features or components disclosed in the specification, and are not intended to preclude the possibility that one or more other features or components may be added.

Sizes of components in the drawings may be exaggerated or reduced for convenience of explanation. In other words, since sizes and thicknesses of components in the drawings are arbitrarily illustrated for convenience of explanation, the following embodiments are not limited thereto.

When a certain embodiment may be implemented differently, a specific process order may be performed differently from the described order. For example, two consecutively described processes may be performed substantially at the same time or performed in an order opposite to the described order.

Here, a psychology discrimination service is a service that provides a psychological state of a user as an object to be examined by connecting the user as an object to be examined and a user as a psychology analysis counselor, and may include a service that automatically calculates a psychological state of an object to be examined based on data generated by the object to be examined. The psychology discrimination service may automatically calculate a psychological state of a user by using a relation between input factors generated by a statistical method and a psychological state as an output.

In addition, the psychology discrimination service may determine a psychological state value by using a model generated by a machine learning algorithm using an artificial neural network. The psychology discrimination service may extract types of input factors presented by a designed training model and used to calculate a psychological state of a user and calculate a psychological state as an output by inputting the extracted input factors to the training model. The training model may output a psychological state as an output by using weight values applied to input factors.

An object to be examined may use a psychology discrimination service by installing a psychological state discrimination unit configured to provide a psychological discrimination service in an electronic device. The psychological state discrimination unit may provide, in addition to a function of discriminating a psychological state of a user, a sharing platform function of sharing input data with other users, a posting function of posting data input by a user, or the like.

A psychology discrimination service may be performed by a program installed on an electronic device, and may be performed in conjunction with an external management server.

An electronic device 100 may discover different and distinguishable patterns according to psychological state information of each user from drawings, signatures, handwritings, or the like of the user by extracting various pieces of data related to a generated touch event (a touch start, a touch movement, a touch end, a stop, a touch cancel, or the like) in units of time in milliseconds or less and arranging an absolute time at which each event occurred on a relative time series from 0 to 100%. A relative time-series transformation pattern for drawing data may be converted into psychological state information of a user who is an object generating the drawing data.

The electronic device 100 may calculate a relationship between visually identical and similar drawing data.

The electronic device 100 may obtain not only the similarity of shapes and/or colors of drawing data, but also a relationship between pieces of digital pattern information of the drawing data. Digital log data of drawing data may include a time taken for drawing, time-series differences in a drawing occupancy space (start and end points within an input unit), the total number of strokes in drawing, a pause pattern in the middle of drawing (break time and time period thereof out of a time taken for drawing), touch pen pressure, a touch movement velocity, information related to eraser usage (a point at which an eraser is used and a rate of the number of erases out of a total number of strokes). In addition, even when there is no difference in color with the naked eye, a red, green, and blue (RGB) ratio, a transparency value, or the like of each touch (pixel) of drawing data may be stored as digital log data.

The digital log data stored in this way may be included in drawing data and may be converted into pattern information according to a preset rule.

The electronic device 100 may determine psychological state values of an original author and an imitator, who generated drawing data, based on digital log data related to shape and color. For example, even when N pieces of drawing data are morphologically and/or chromatically similar, digital pattern information of the N pieces of drawing data may be different. Digital pattern information of drawing data may be difficult to confirm with the naked eye. A psychological state value of a user of each piece of drawing data may be determined by considering digital pattern information of the drawing data. The electronic device 100 may analyze psychological states of an imitator and an original author by classifying drawings of the imitator and the original author into an imitation data group and calculating relative pattern information and a relative psychological state value between pieces of drawing data in the imitation data group.

In addition, the electronic device 100 may discriminate the psychological states of the imitator and the original author by using a training model trained by using the drawing data of the imitation data group and the relative pattern information between pieces of the drawing data as an input and using the relative psychological state value as an output.

Here, the training model uses a machine learning algorithm, and may include one or more artificial neural networks to determine constant parameters applied to input variables in outputting output variables from the input variables. In particular, supervised training, unsupervised training, and reinforcement training methods may be used, but the present disclosure is not limited thereto, and various machine training methods may be used.

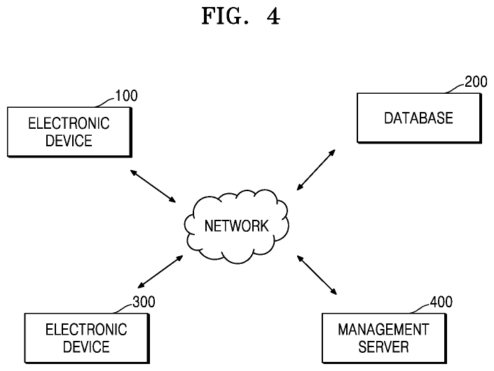

Generation of a training model may be performed within the electronic device 100 or may be performed by the management server 400 (in FIG. 4) in the outside.

In the present disclosure, a touch event refers to a point of touching a digital device with a finger or a touch pen. For a touch start event, drawing data, such as coordinate values, a time value, a diameter of a touch point, a color of a point, a pen pressure value, a stop time value, a touch inclination value, or the like, may be extracted.

In the present disclosure, a touch movement event refers to a trajectory of touch points from a start of a touch until an end of the touch. The drawing data for this may be a combination of a plurality pieces of movement data based on the inclination of the trajectory or a moved distance. In addition, the drawing data may include coordinate values, elapsed time, a movement distance, a velocity value, a color of a trajectory, a thickness of a trajectory, a change value of touch pen pressure compared to initial touch pen pressure, a change in touch trajectory, or the like.

In the present disclosure, a touch end event refers to a point at which a touch is ended or a point at which a touch of a stopped finger or touch pen is dropped. After the touch end event is made, a touch start event may be repeated again after stopping.

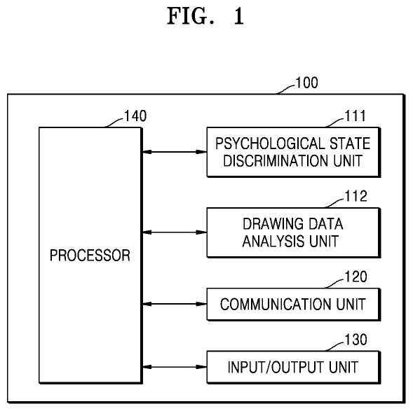

FIG. 1 is a block diagram of an electronic device 100 according to embodiments of the present disclosure.

The electronic device 100 may include a psychological state discrimination unit 111, a drawing data analysis unit 112, a communication unit 120, an input/output unit 130, and a processor 140.

The psychological state discrimination unit 111 and/or the drawing data analysis unit 112 may be a set of one or more instructions. The psychological state discrimination unit 111 and/or the drawing data analysis unit 112 may be implemented as a computer-readable medium. The psychological state discrimination unit 111 and/or the drawing data analysis unit 112 may be random access memory (RAM), read only memory (ROM), or a permanent mass storage device such as a disk drive. The psychological state discrimination unit 111 and/or the drawing data analysis unit 112 may be a computer-readable recording medium such as a floppy drive, a disk, a tape, a digital video disc (DVD)/compact disc (CD)-ROM drive, a memory card, or the like.

The communication unit 120 may provide a function of communicating with an external device through a network. For example, a request generated by the processor 140 of the electronic device 100 according to program code stored in a recording device such as the psychological state discrimination unit 111 may be transmitted to an electronic device 300 (in FIG. 4), a database 200 (in FIG. 4), or a management server 400 (in FIG. 4) via a network under the control by the communication unit 120. For example, a control signal or a command received through the communication unit 120 may be transmitted to the processor 140 or a storage medium, the psychological state discrimination unit 111, and/or the drawing data analysis unit 112, and a received video image or the like may be stored in a storage medium, the psychological state discrimination unit 111, and/or the drawing data analysis unit 112.

The input/output unit 130 may display a screen providing information, or may receive an input from a user. For example, the input/output unit 130 may include an operation panel receiving a user input, a display panel displaying a screen, or the like.

In particular, the input/output unit 130 may include devices which may receive various types of user inputs, such as a keyboard, a physical button, a touch screen, a camera, or a microphone. Also, the input/output unit 130 may include a display panel, a speaker, or the like. However, the present disclosure is not limited thereto, and the input/output unit 130 may include a configuration supporting various inputs/outputs.

The processor 140 may be implemented by one or more processors, and may be configured to process a command of a computer program by performing basic calculation, logic, and input/output operations. A command may be provided to the processor 140 by a storage medium and the communication unit 120. For example, the processor 140 may be configured to execute a command received according to program code stored in the psychological state discrimination unit 111 and/or the drawing data analysis unit 112, or a recording device such as a storage medium.

The electronic device 100 may further include a computer-readable recording medium such as RAM and ROM, and a permanent mass storage device such as a disk drive.

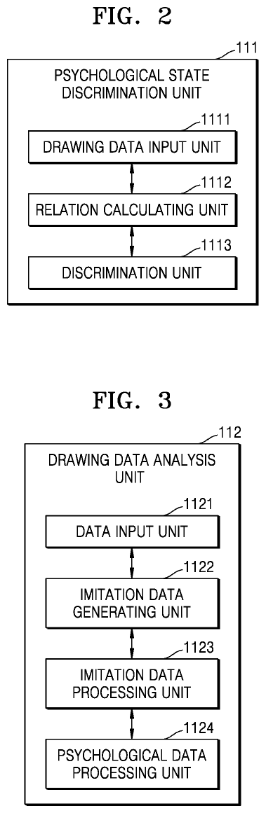

FIG. 2 is a block diagram of the psychological state discrimination unit 111 according to embodiments of the present disclosure.

The psychological state discrimination unit 111 may include a drawing data input unit 1111, a relation calculating unit 1112, and a discrimination unit 1113.

The drawing data input unit 1111 may be input feature values of touch events generated by a touch input unit with a touch unit.

The relation calculating unit 1112 may calculate drawing pattern information of a user by using the feature values of the touch events. Here, the drawing pattern information may include a certain rule in drawing data. The relation calculating unit 1112 may calculate pattern information such as a pattern over time of accumulated distances and velocity values of touch events of drawing data, a pattern over time of a total distance and velocity values of touch events, a change pattern of accumulated time and stop time values, a change pattern of relative pen pressure, occupancy rate values, distribution of colors, and change pattern of a usage time for each color, or the like. Here, the occupancy rate value may be calculated based on occupied and non-occupied pixels in a drawing space. In particular, the occupancy rate value of the non-occupied pixels may be calculated based on a pen pressure value, a color value, a distance value, and an occupancy time value of an adjacent occupancy level. In an optional embodiment, the occupancy rate value of the non-occupied pixels may be determined based on an occupancy rate value of an adjacent occupancy level. Here, a change pattern of pattern information may refer to a rule that changes according to the passage of time or a drawing process.

In addition, the relation calculating unit 1112 may calculate pattern information, by using feature values of touch events. The pattern information may be a change pattern of coordinate values and pen pressure values, a change pattern of coordinate values and diameter values of points, a change pattern of inclination values and distance values or velocity values of a touch unit, a change pattern of inclination values and distance values of a trajectory, a relative change pattern between inclination values and velocity values of a trajectory, a pattern in which a stop section occurs in correspondence to a time taken for drawing, a change pattern between movement distance values and stroke thickness values of each touch, a change pattern between distance values and stroke thickness values for a reference point of each touch, information about a ratio between each of color values, or the like. Here, a change pattern of pattern information may refer to a rule in which two data values change over time or a rule in which two data values change during a drawing process.

However, the relation calculating unit 1112 is not limited thereto, and may calculate various types of pattern information. The relation calculating unit 1112 may convert behavior data, input data, setting data, or the like of a user into pattern information. Here, pattern information refers to a certain rule, and may include a rule that changes over time or a rule that changes while using an electronic device. The behavior data is data related to a behavior of a user using the electronic device 100, and may include screen activation-related information (time point, number, etc.), execution-related data of other applications (frequency of execution, execution frequency cycle), the number of applications being executed in a background, whether applications being executed in a background are activated, or the like. The input data is data related to input values, and may include data of setting values for other applications and data of basic setting values of a system of the electronic device 100. The setting data may include an enrollment path, an enrollment application, an enrollment date, an enrollment region, time of payment, profile registration information, an access device, an access date, access time (time of day, week, and month), access frequency (daily, weekly, and monthly frequency), an access location (street name, an accumulated distance traveled between access locations, etc.), an access weight (emotional word selection rate information, search rate information, drawing rate information, touch rate information, alarm confirmation rate, monthly report, etc.), an access environment, environment setting information, or the like.

The discrimination unit 1113 may discriminate a psychological state value of a user by using pattern information calculated by the relation calculating unit 1112.

Here, the psychological state value may be set as a state value for each type such as openness, conscientiousness, extraversion, agreeableness, neuroticism, stamina, etc., but is not limited thereto, and may be set as state values for each of various types.

The discrimination unit 1113 may determine a psychological state value of a user by using a relation or a table corresponding to values of the pattern information calculated by the relation calculating unit 1112 and the types of psychological state values.

FIG. 3 is a block diagram of the drawing data analysis unit 112 according to embodiments of the present disclosure.

The drawing data analysis unit 112 may include a data input unit 1121, an imitation data generating unit 1122, an imitation data processing unit 1123, and a psychological data processing unit 1124.

The data input unit 1121 may obtain, from the psychological state discrimination unit 111, drawing data, pattern information corresponding to the drawing data, and a psychological state value according to the pattern information. The pattern information may include a change pattern of velocity values for each accumulated movement distance and time, a change pattern of accumulated time and stop time values, a change pattern of relative pen pressure, a change pattern of occupancy rate values, a change pattern of color distribution, a change pattern of usage time for each color, a relative change pattern between coordinate values and pen pressure values, a change pattern of coordinate values and diameter values (thickness values) of a touch point, a change pattern of inclination values and distance values of a touch unit, a change pattern of inclination values and velocity values of a touch unit, a change pattern of inclination values and distance values of a trajectory, a change pattern of inclination values and velocity values of a trajectory, a pattern in which a stop section occurs in correspondence to a time taken for drawing, a change pattern of distance values and stroke (touch) thickness values of each touch, or a pattern of ratio information between each of color values, which are calculated by the relation calculating unit 1112, but is not limited thereto, and may include other patterns. Pattern information may be set with a certain relation or may be set with a certain table. Here, a change pattern of the pattern information may refer to a rule that changes according to the passage of time or a drawing process.

The data input unit 1121 may be connected to drawing data generated by a corresponding electronic device to receive imitated drawing data. The imitated drawing data may be received from the management server 400 (in FIG. 4) or another electronic device.

The imitation data generating unit 1122 may generate externally similar pieces of data among input pieces of drawing data as an imitation data group.

The imitation data generating unit 1122 may classify a first piece of drawing data and a second piece of drawing data, which are similar to each other among the input pieces of drawing data, into an imitation data group. The imitation data generating unit 1122 may repeatedly extract imitation data groups existing in drawing data.

The imitation data generating unit 1122 may extract one or more first objects in the first piece of drawing data and one or more second objects in the second piece of drawing data, and calculate a degree of similarity between the first object and a corresponding second object. The imitation data generating unit 1122 may classify the first piece of drawing data and the second piece of drawing data into an imitation data group by determining whether a degree of similarity thereof is greater than or equal to a preset minimum similarity value. The minimum similarity value may be a value changeable by a training algorithm or an administrator.

The degree of similarity between pieces of drawing data is comprehensively determined by a degree of morphological similarity, a degree of color similarity, or the like, and a method of determining a degree of similarity may use a method of determining a degree of similarity between general images. For example, methods such as feature matching, histogram, mean square error (MSE), and autoencoder may be used.

The imitation data generating unit 1122 may designate drawing data of each of an imitator and an original author within an imitation data group, and may give an index corresponding to an imitation order between the drawing data. Here, the imitator refers to a user who is not an original author. Drawing data of an original author may be determined by a creation time point of corresponding data, a final storage time point, or the like. An original author may be determined by determining whether drawing data is from the original author.

Optionally, the drawing data of the original author may be determined by using pattern information of the drawing data. For example, when the first piece of drawing data has pattern information that is not similar to average pattern information of a first user who is a creator, the first piece of drawing data may be determined as being drawn, that is, imitated, according to other drawing data. Alternatively, when the pattern information of the first piece of drawing data is similar to drawing pattern information of the first user who is the creator, the first piece of drawing data may be determined to be imitated. For each user, two pieces of drawing pattern information may be generated. The first piece of pattern information in the case of imitation and the second piece of pattern information in the case of non-imitation may be generated for each user. When the first piece of pattern information and the second piece of pattern information differ from each other by a certain degree or less, whether the drawing data of the first user is imitated may not be determined by pattern information. When the first piece of pattern information and the second piece of pattern information differ from each other by a certain degree or more, whether the drawing data of the first user is imitated may be determined by the pattern information. Also, when the second piece of drawing data has pattern information similar to average pattern information of the second user who is a creator, the second piece of drawing data may be determined to be original, but is not limited thereto.

The imitation data processing unit 1123 may generate relative pattern information by loading one or more pieces of drawing data in an imitation data group and comparing one or more pieces of pattern information corresponding to the one or more pieces of drawing data. The imitation data processing unit 1123 may store the generated relative pattern information in correspondence with the imitation data group. Here, the relative pattern information refers to including difference values between pattern values.

More particularly, when the first piece of drawing data and the second piece of drawing data are in the imitation data group, the imitation data processing unit 1123 may load the first piece of drawing data and the second piece of drawing data and compare a first piece of pattern information corresponding to the first piece of drawing data with a second piece of pattern information of the second piece of drawing data to convert the second piece of pattern information into relative pattern information compared with the first piece of pattern information and store the first piece of pattern information, the second piece of pattern information, and the relative pattern information in correspondence with the imitation data group. The relative pattern information may include a difference value between the first piece of pattern information and the second piece of pattern information, but is not limited thereto.

The imitation data processing unit 1123 may repeatedly perform the same operation as the above method to determine imitation data groups corresponding to generated drawing data, and generate pieces of relative pattern information related to each of the imitation data groups to store the generated relative pattern information in correspondence to each imitation data group.

The imitation data processing unit 1123 may generate relative pattern information based on original drawing data. In addition, the imitation data processing unit 1123 may perform indexing according to an imitation order to generate relative pattern information for the indexed drawing data.

The psychological data processing unit 1124 may generate data for an imitation data group. The psychological data processing unit 1124 may use drawing data, pattern information of the drawing data, relative pattern information, index information based on an imitation order, a distinction between an original author and an imitator, and information on an imitation order among imitators, which belong to the imitation data group, to determine a psychological state value and/or a psychological relation of creators of the drawing data.

The imitation data processing unit 1123 may calculate psychological state values of creators and a relation between the creators from the drawing data of an imitation data group by using a model trained by machine learning. The training model may be trained by using digital log data, pattern information thereof, and relative pattern information from the drawing data as an input, and using a psychological state value and a psychological correlation as an output. The training model may be completed by determining types of factors that have a high correlation in calculation of output variables from the digital log data, the pattern information thereof, and relative pattern information through an iterative learning operation using an artificial neural network and increasing the reliability of outputting output variables with these input variables.

FIG. 4 is a diagram of a psychology discrimination system according to embodiments of the present disclosure.

The psychology discrimination system may include the electronic device 100 carried by users, the electronic device 300, the database 200 managing data for psychology discrimination, and the management server 400.

The electronic device 100 and/or the electronic device 300 may be installed with the psychological state discrimination unit 111 and/or the drawing data analysis unit 112. A user may execute the psychological state discrimination unit 111 to input drawing data and receive a psychological state value corresponding to the drawing data. The drawing data analysis unit 112 may generate an imitation data group from among the pieces of drawing data obtained through the psychological state discrimination unit 111, and may generate pattern information and relative pattern information for each imitation group. The electronic device 100 or the electronic device 300 may link the drawing data, the pattern information, the psychological state value, or the like obtained from the psychological state discrimination unit 111 for each user and transmit the same to the database 200. The electronic device 100 and/or the electronic device 300 may generate an imitation data group and transmit digital log data, pattern information, relative pattern information, or the like for each imitation data group to the database 200.

When there is a user's action or input, the psychological state discrimination unit 111 in the electronic device 100 generates data in response to the user's action or input. Data generated in response to the user's action or input may include a timestamp value at a time point of the action, a timestamp value at a time point of input, an environment information value at the time point of the action, an environment information value at the time point of input, or the like. Data generated in response to the user's actions or input may be converted into values by a predefined table.

The management server 400 may receive data from the electronic devices 100 and 300 and/or the database 200 through a network. The management server 400 may generate a conversion expression of data generated in response to the user's action or input, and distribute the generated conversion expression to the electronic devices 100 and 300. The management server 400 may manage information related to discrimination of a psychological state in association with pattern information and relative pattern information in the imitation data group.

The management server 400 may update the psychological state discrimination unit 111 to collect only data or factors whose degree of relation to the discrimination of a psychological state is greater than or equal to a present basic discrimination level.

The management server 400 may update the drawing data analysis unit 112 by updating a logic that generates an imitation data group.

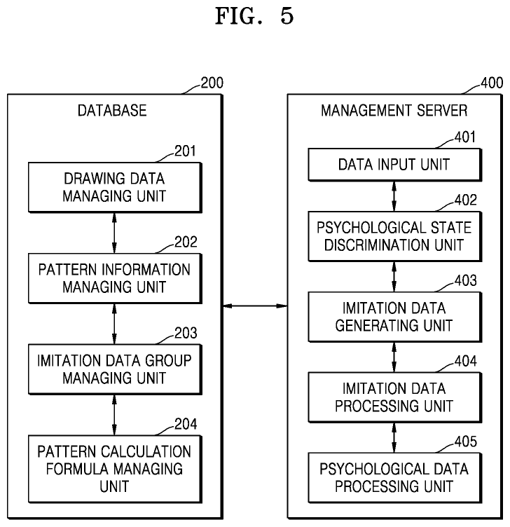

FIG. 5 is a block diagram of the database 200 and/or the management server 400 and shows an operation of data transmission/reception.

The database 200 and the management server 400 are computing devices having one or more processors, and may include a communication module for communication with an external device, an input device for data input and output, or an output device.

The database 200 may receive data from one or more electronic devices 100 and 300, which are connected to the database 200. The database 200 may include a drawing data managing unit 201 managing drawing data and digital log data received from one or more electronic devices 100 and 300, a pattern information managing unit 202 managing pattern information calculated in response to drawing data, an imitation data group managing unit 203, and a pattern calculation formula managing unit 204.

The pattern calculation formula managing unit 204 may manage a calculation formula (or table) that generates pattern information by inputting feature values of drawing data, or a calculation formula (or table) for calculating a psychological state value by inputting pattern information, or the like.

The pattern calculation formula managing unit 204 may receive and store a training model generated by the management server 400 or the electronic devices 100 and 300.

The management server 400 may include a data input unit 401, a psychological state discrimination unit 402, an imitation data generating unit 403, an imitation data processing unit 404, and a psychological data processing unit 405. The data input unit 401 may input data from the electronic devices 100 and 300 and/or the database 200. The imitation data generating unit 403 may generate an imitation data group based on the input data. The imitation data processing unit 404 may generate pattern information for each imitation data group. The psychological data processing unit 405 may convert the generated pattern information for each imitation data group into relative pattern information and determine psychological state-related data based on the relative pattern information for each imitation data group.

The data input unit 401 may obtain drawing data, pattern information corresponding to the drawing data, and/or a psychological state value according to the pattern information from the electronic devices 100 and 300 and/or the database 200. The pattern information may include a change pattern of an accumulated distance or a total distance and velocity values over time, a change pattern of accumulated time and stop time values, a change pattern of relative pen pressure, a change pattern of occupancy rate values, a change pattern of color distribution, a change pattern of usage time for each color, a change pattern of coordinate values and pen pressure values over time, a change pattern of coordinate values and diameter values (thickness) of a touch, a change pattern of inclination values and distance values of a touch unit, a change pattern of inclination values and velocity values of a touch unit, a change pattern of inclination values and distance values of a trajectory, a change pattern of inclination values and velocity values of a trajectory, a pattern in which a stop section occurs in correspondence to a time taken for drawing, a change pattern of distance values and stroke thickness values of each touch, a change pattern of ratio information between each of color values, or the like. Here, a change pattern of pattern information may refer to a rule that changes according to the passage of time or a drawing process.

The psychological state discrimination unit 402 may calculate drawing pattern information of a user by using feature values of touch events. The psychological state discrimination unit 402 may calculate pattern information such as a change pattern of accumulated distance and velocity values of touch events of drawing data, a change pattern of accumulated time and stop time values, a change pattern of relative pen pressure, a change pattern of occupancy rate values, a change pattern of color distribution, a change pattern of usage time for each color, or the like.

In addition, the psychological state discrimination unit 402 may calculate pattern information, by using the feature values of the touch events, such as a change pattern of coordinate values and pen pressure values, a change pattern of coordinate values and diameter values (thickness values) of a touch point, a change pattern of inclination values and distance values of a touch unit, a change pattern of inclination values and velocity values of a touch unit, a change pattern of inclination values and distance values of a trajectory, a change pattern of inclination values and velocity values of a trajectory, a change pattern in which a stop section occurs in correspondence to a time taken for drawing, a change pattern of distance values and stroke thickness values of each touch, ratio information between each of color values, a change pattern of ratio information, or the like. Here, a change pattern of pattern information may refer to a rule that changes according to the passage of time or a drawing process.

However, the psychological state discrimination unit 402 is not limited thereto, and may calculate various patterns. Here, pattern information refers to a certain rule, and may include a rule that changes over time or a rule that changes while using an electronic device. The psychological state discrimination unit 402 may convert behavior data of a user, input data, setting data, or the like into pattern information. The behavior data is data related to a behavior of a user using the electronic device 100, and may include screen activation-related information (time, number, etc.), execution-related data of other applications (frequency of execution, execution frequency cycle, etc.), the number of applications being executed in a background, whether applications being executed in a background are activated, or the like. The input data is data related to input values, and may include data of setting values for other applications and data of basic setting values of a system of the electronic device 100. The setting data may include an enrollment path, an enrollment application, an enrollment date, an enrollment region, time of payment, profile registration information, an access device, an access date, access time (time of day, week, and month), access frequency (daily, weekly, and monthly frequency), an access location (street name, cumulative distance traveled between access locations, etc.), an access weight (emotional word selection rate information, search rate information, drawing rate information, touch rate information, alarm confirmation rate, monthly report, etc.), an access environment, environment setting information, or the like, which are related to an enrollment behavior of a user, but is not limited thereto.

The psychological state discrimination unit 402 may determine a psychological state value of a user by using the calculated pattern information.

The psychological state discrimination unit 402 may determine a psychological state value of a user by using a relation or a table corresponding to values of the calculated pattern information and the types of psychological state values.

The data input unit 401 may obtain, from the psychological state discrimination unit 402, drawing data, pattern information corresponding to the drawing data, and a psychological state value according to the pattern information.

The pattern information may include a change pattern of accumulated distance or a total distance and velocity values over time, a change pattern of accumulated time and stop time values, a change pattern of relative pen pressure, a change pattern of occupancy rate values, a change pattern of color distribution, a change pattern of usage time for each color, a change pattern of coordinate values and pen pressure values, a change pattern of coordinate values and diameter values (thickness values) of a touch point, a change pattern of inclination values and distance values of a touch unit, a change pattern of inclination values and velocity values of a touch unit, a change pattern of inclination values and distance values of a trajectory, a change pattern of inclination values and velocity values of a trajectory, a pattern in which a stop section occurs in correspondence to a time taken for drawing, a change pattern of distance values and stroke thickness values of each touch, ratio information between each of color values, or the like, but is not limited thereto. The change pattern of accumulated distance may be a change pattern of a movement accumulated distance of a touch during drawing. A total distance over time refers to the change pattern of the total distance traveled. Velocity values over time refer to the change pattern of the touch velocity value.

The change pattern of stop time values includes pattern data for time values at which drawing is stopped. The change pattern of relative pen pressure includes changing pattern data of pen pressure values of touch during drawing. The change pattern of occupancy rate values includes pattern data in which the occupancy rate changes over time. The change pattern of color distribution may include distribution ratio values of color values for each time. The change pattern of usage time for each color may include pattern data on usage of each color for each time. The change pattern of coordinate values may include pattern data in which coordinate values of each touch change over time.

The imitation data generating unit 403 may generate externally similar pieces of data among input pieces of drawing data as an imitation data group. An imitation data group may be generated based on drawing data in which an imitation relation is set by a user.

The imitation data generating unit 403 may classify a first piece of drawing data and a second piece of drawing data, which are similar to each other among the input pieces of drawing data, into an imitation data group. The imitation data generating unit 403 may repeatedly extract imitation data groups existing in drawing data.

The imitation data generating unit 403 may extract one or more first objects in the first piece of drawing data and one or more second objects in the second piece of drawing data, and calculate a degree of similarity between the first object and a corresponding second object. The imitation data generating unit 403 may classify the first piece of drawing data and the second piece of drawing data into an imitation data group by determining whether a degree of similarity thereof is greater than or equal to a preset minimum similarity value.

The degree of similarity between pieces of drawing data is comprehensively determined by a degree of morphological similarity, a degree of color similarity, or the like, and a method of determining a degree of similarity may use a method of determining a degree of similarity between general images.

The imitation data generating unit 403 may designate drawing data of an imitator and an original author within an imitation data group, and may give an index corresponding to an imitation order between the drawing data. Drawing data of an original author may be determined by a creation time point of corresponding data, a final storage time point, or the like. Optionally, the drawing data of the original author may be determined by using pattern information of the drawing data. For example, when the first piece of drawing data has pattern information that is not similar to average pattern information of a first user who is a creator, the first piece of drawing data may be determined to be imitated. This is because, in the act of imitation, the drawing may be made with a pattern different from the one's own pattern information. These imitation-related characteristics may be determined based on each user's drawing pattern information. Alternatively, when the pattern information of the first piece of drawing data is similar to drawing pattern information of the first user who is the creator, the first piece of drawing data may be determined to be imitated.

Also, when the second piece of drawing data has pattern information similar to average pattern information of the second user who is a creator, the second piece of drawing data may be determined to be original, but is not limited thereto.

The imitation data generating unit 403 may generate relative pattern information by loading one or more pieces of drawing data in an imitation data group and comparing one or more pieces of pattern information corresponding to the one or more pieces of drawing data. The generated relative pattern information may be stored in correspondence with the imitation data group.

More particularly, when the first piece of drawing data and the second piece of drawing data are in the imitation data group, the imitation data generating unit 403 may load the first piece of drawing data and the second piece of drawing data and compare a first piece of pattern information corresponding to the first piece of drawing data with a second piece of pattern information of the second piece of drawing data to convert the second piece of pattern information into relative pattern information compared with the first piece of pattern information and store the first piece of pattern information, the second piece of pattern information, and the relative pattern information in correspondence with an imitation data group.

The imitation data processing unit 404 may repeatedly perform the same operation as the above method to determine imitation data groups corresponding to generated drawing data, and generate pieces of relative pattern information related to each of the imitation data groups to store the corresponding pattern information in correspondence to each imitation data group.

The imitation data processing unit 404 may generate relative pattern information based on original drawing data. In addition, the imitation data processing unit 404 may perform indexing according to the imitation order to generate relative pattern information.

The psychological data processing unit 405 may generate data for an imitation data group. The psychological data processing unit 405 may use drawing data, pattern information of the drawing data, relative pattern information, index information based on an imitation order, a distinction between an original author and an imitator, and information on an imitation order among imitators, which belong to an imitation data group, to determine a psychological value and/or psychological correlation of creators of the drawing data.

The imitation data processing unit 404 may calculate psychological state values of creators and a correlation between the creators from the drawing data of an imitation data group by using a model trained by machine learning. The training model may be trained by using digital log data, pattern information thereof, and relative pattern information from the drawing data as an input, and using a psychological state value and a psychological correlation as an output. The training model may be completed by determining types of factors that have a high correlation in calculation of output variables from the digital log data, the pattern information thereof, and relative pattern information through an iterative training operation using an artificial neural network and increasing the reliability of outputting output variables with these input variables.

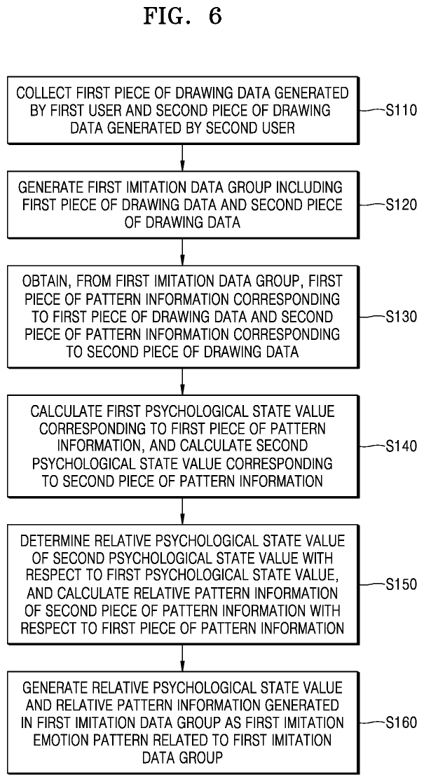

FIG. 6 is a flowchart of a method of generating relative pattern information between imitation drawing data, according to embodiments of the present disclosure.

In S110, the management server 400 may collect a first piece of drawing data generated by a first user and a second piece of drawing data generated by a second user.

In S120, the management server 400 may generate a first imitation data group including the first piece of drawing data and the second piece of drawing data.

In S130, the management server 400 may obtain, from the first imitation data group, a first piece of pattern information corresponding to the first piece of drawing data and a second piece of pattern information corresponding to the second piece of drawing data.

In S140, the management server 400 may calculate a first psychological state value corresponding to the first piece of pattern information, and calculate a second psychological state value corresponding to the second piece of pattern information.

In another embodiment, in relation to S130 and S140, the management server 400 may receive, from an electronic device of the first user, the first piece of pattern information corresponding to the first piece of drawing data and the first psychological state value corresponding to the first piece of pattern information. The management server 400 may receive, from an electronic device of the second user, the second piece of pattern information corresponding to the second piece of drawing data and the second psychological state value corresponding to the second piece of pattern information.

In S150, the management server 400 may determine a relative psychological state value of the second psychological state value with respect to the first psychological state value, and calculate relative pattern information of the second piece of pattern information with respect to the first piece of pattern information.

In another embodiment, the management server 400 may determine the relative psychological state value by using the received first psychological state value and the second psychological state value, and calculate the relative pattern information by using the first piece of pattern information and the second piece of pattern information.

In S160, the management server 400 may generate the relative psychological state value and the relative pattern information generated in the first imitation data group as a first imitation emotion pattern related to the first imitation data group. The management server 400 may generate the first imitation emotion pattern based on an index based on an imitation order of one or more pieces of drawing data of the first imitation data group.

The first imitation emotion pattern may be generated according to an index based on an imitation order, based on a psychological state value of an original author.

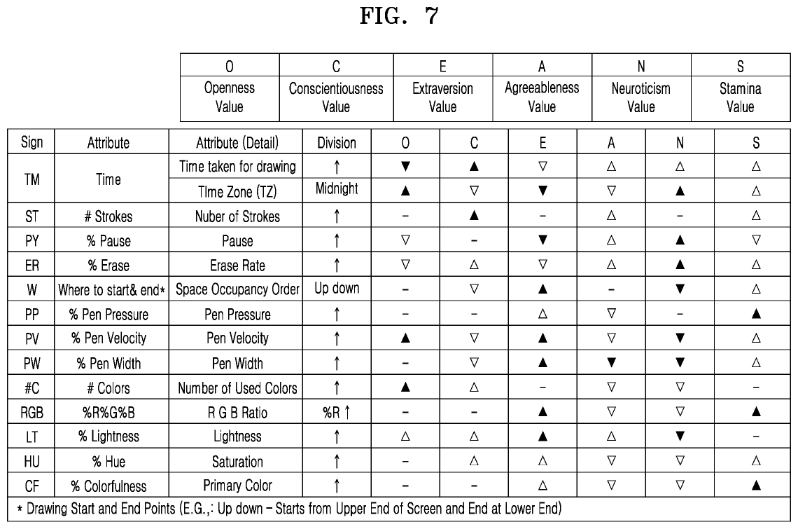

FIG. 7 shows an example of a correlation table showing pattern information used for psychological state discrimination and each type of psychological state value.

The correlation table refers to a correlation coefficient between drawing data and psychological state value. The correlation table may include a correlation coefficient between two selected values, a fact whether the correlation coefficient is proportional, or a relation. The drawing data includes a time taken for drawing, a drawing time zone, the number of touches in drawing, a ratio of stop time/number of times during drawing, a ratio of erase time/number of times during drawing, a space occupancy value of drawing data, a relative pen pressure value converted based on a maximum value, a relative velocity value converted based on a maximum value, a relative thickness value, the number of color values used, ratio information of used time/number of times of color values, a ratio of brightness values, a ratio of saturation values, and a ratio of primary color values in the drawing. The time taken for drawing is the time required to generate drawing data. The drawing time zone is the time zone to generate drawing data. The number of touches in drawing is the number of touch inputs included in the drawing data. The stop ratio during drawing is a percentage of time that drawing was stopped during drawing. The erase ratio during drawing is a percentage of time or number of times an erase input is used during drawing. The space occupancy value of drawing data is a value for the space occupied by the drawing in the entire drawing space. The space occupancy value may be determined in consideration of a ratio of a space drawn in the entire space, the number of times each pixel is drawn, and the like.

The relative pen pressure value is the pen pressure values of touches for which drawing data are generated, and may be a relative value in contrast to the largest pen pressure value. The relative velocity value may be a velocity value at which touches are moved when drawing is generated. The relative velocity value may be a relative value compared to the maximum velocity value. The relative thickness value may be a thickness value of a touch generating drawing data. The relative thickness value may be a thickness value compared to the maximum thickness value. The number of color values used may be the number of colors used for drawing. The ratio information of color values may be a ratio value of each color used corresponding to the entire drawing. The ratio of brightness values may be a ratio value of each brightness value to brightness values used in the entire drawing. The ratio of saturation values may be a ratio value of each saturation value to saturation values used in the entire drawing. The ratio of primary color values in the drawing may be a ratio value of each primary color value to primary color values used in the entire drawing. The psychological state values includes an openness value (O), a conscientiousness value (C), an extraversion value (E), an agreeableness value (A), a neuroticism value (N), and a stamina value (S).

The correlation table is generated by the psychological state discrimination unit 402 of the management server 400, and may be transmitted from the management server 400 to the electronic devices 100 and 300.

The psychological state discrimination unit 111 of each electronic device may determine a psychological state value by using the correlation table generated as described above. In addition, the psychological state discrimination unit 111 may determine input variables of the correlation table, the input variables including a time taken for drawing, a drawing time zone, the number of touches in a drawing, a stop ratio in drawing, an erase ratio in drawing, a space occupancy value of drawing data, a relative pen pressure value, a relative velocity value, a relative thickness value, the number of color values used, ratio information of color values, a ratio of a brightness value, a ratio of a Chroma value, and ratio information of primary color values in the drawing.

Thirteen kinds of drawing data may correspond to values for each type of psychological state value. The psychological state value relates to the user who generated the drawing data.

TM (Time, time taken for drawing)—in relation to a total drawing time, when drawing time is long, openness (O) of the psychological state value is low and conscientiousness (C) of the psychological state value is high.

TZ (Time zone)—in relation to a drawing time zone, openness (O) of the psychological state value and neuroticism (N) of the psychological state value are high, and extroversion (E) of the psychological state value is low in a midnight time zone.

ST (Number of strokes)—in relation to the total number of touch strokes for drawing, when the number of touch strokes is high, conscientiousness (C) of the psychological state value is high.

PU (Pause, pause pattern)—in relation to the frequency and duration of pauses in a total drawing time, extroversion (E) of the psychological state value is low when the time of pauses is long, and stamina (S) of the psychological state value is low when the frequency of pauses is frequent.

ER (Erase, erase pattern)—in relation to the rate and frequency of erasing out of the total number of strokes for drawing, when an erase rate is high, neuroticism (N) of the psychological state value is high.

W (Where to start and end, space occupancy order)—refers to where (top, bottom, left, and right) drawing starts and where drawing ends in a space on a canvas, when a drawing start point is on the top of the canvas, extroversion (E) of the psychological state value is high.

PP (Pen pressure)—in relation to a pen pressure level, when pen pressure is high, stamina (S) of the psychological state value is high.

PV (Pen velocity)—in relation to a touch stroke velocity, when a touch stroke velocity is fast, openness (O) of the psychological state value and extraversion (E) of the psychological state value are high, and neuroticism (N) of the psychological state value is low.

PW (Pen width)—in relation to a touch stroke thickness, when a thick touch stroke is used, extraversion (E) of the psychological state value is high, and agreeableness (A) of the psychological state value and neuroticism (N) of the psychological state value are low.

#C (Number of colors)—when the total number of colors used is large and the number of colors is large, openness (O) of the psychological state value is high.

RGB (RGB ratio)—when a weight of red (R) of the psychological state value is high, extraversion (E) and stamina (S) of the psychological state value are high.

LT (Lightness)—in relation to brightness of a drawing, when a brightness is high, extraversion (E) of the psychological state value is high, and neuroticism (N) of the psychological state value is low.

HU (Hue)—in relation to a saturation of a drawing, when a saturation is high, conscientiousness (C) and extraversion (E) of the psychological state value are high, and neuroticism (N) of the psychological state value is low.

CF (Colorfulness)—when a primary color sense of a drawing is high and a weight of primary colors is high, stamina (S) of the psychological state value is high.

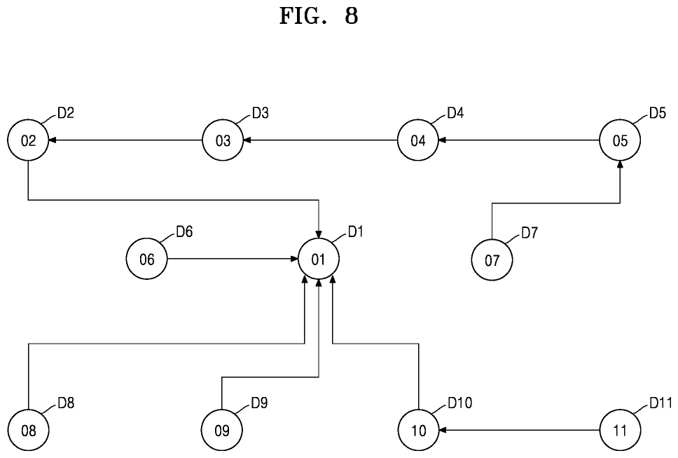

FIG. 8 shows an example of an imitation order of first to eleventh pieces of drawing data of an imitation data group. The imitation data group includes data related to the imitators, and may include data about the imitators and drawing data generated by the imitators.

The management server 400 may receive first piece of drawing data D1 to eleventh piece of drawing data D11 from electronic devices (i.e., the first piece of drawing data D1, a second piece of drawing data D2, a third piece of drawing data D3, a fourth piece of drawing data D4, a fifth piece of drawing data D5, a sixth piece of drawing data D6, a seventh piece of drawing data D7, an eighth piece of drawing data D8, a ninth piece of drawing data D9, a tenth piece of drawing data D10, and the eleventh piece of drawing data D11). The management server 400 may generate an imitation data group for the first piece of drawing data D1 to the eleventh piece of drawing data D11.

As shown in FIG. 8, an imitation relationship may be complicatedly entangled, and data obtained by imitating again imitated data may be generated. Regarding this, a relationship between a relative original author and a relative imitator may be set.

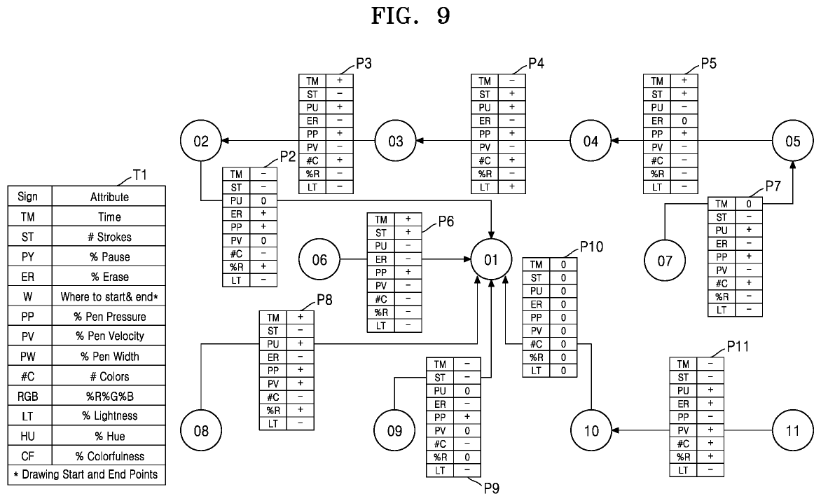

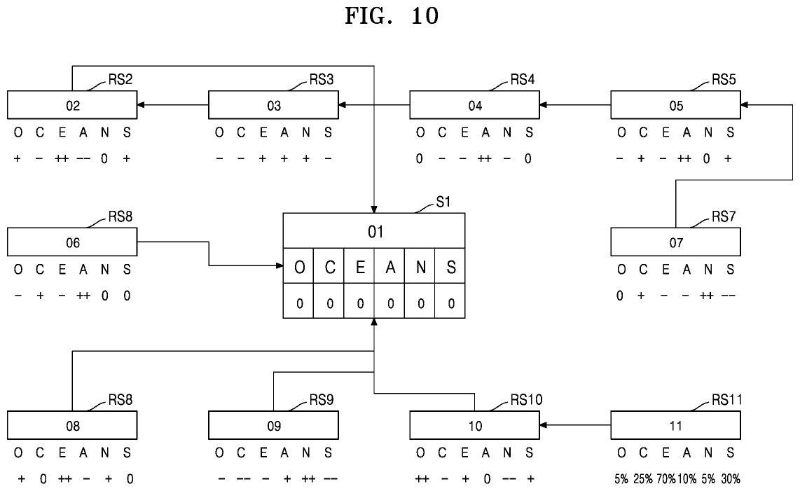

FIG. 9 shows an example illustrating a relative relationship between data values of the first piece of drawing data D1 to the eleventh piece of drawing data D11 of an imitation data group. The management server 400 may vectorize pattern information and psychological values of drawing data into one matrix, and generate these matrices for each imitation relationship to generate an imitation emotion pattern.

The management server 400 may receive first piece of pattern information to eleventh piece of pattern information respectively corresponding to the first piece of drawing data D1 to the eleventh piece of drawing data D11, and generate the second piece of pattern information to the eleventh piece of pattern information as pieces of relative pattern information P2, P3, P4, P5, P6, P7, P8, P9, P10, and P11 corresponding to the first piece of pattern information. The pieces of relative pattern information P2, P3, P4, P5, P6, P7, P8, P9, P10, and P11 may include difference values from the first piece of pattern information of the first piece of drawing data. Types of pattern information used to calculate a psychological state value may be the same as T1.

Pieces of psychological pattern information are relative to the first piece of pattern information, relative to the second piece of pattern information, relative to the third piece of pattern information, and relative to the fourth piece of pattern information, and may be converted into pieces of relative pattern information. The number of pieces of relative pattern information generated from eleven pieces of drawing data may be 11×10/2=55.

When an imitation data group is not considered, for example, eleven pieces of pattern information are generated in correspondence to generated drawing data, but when an imitation data group is considered, even relative pattern information is generated, and an amount of data used for analysis may increase.

The management server 400 may determine one or more users of the imitation data group and one or more psychological state values by using the pattern information and the relative pattern information, which are used for analysis.

The relative pattern information may be set as whether each piece of pattern information is increased or decreased, and may be expressed as, for example, +, −, 0, or a particular numerical value or percentage.

State pattern information of imitated drawing data may be expressed in an index order based on an imitation order.

FIG. 10 shows an example of psychological state values from first to eleventh pieces of drawing data of an imitation data group (S1, RS2, RS3, RS4, RS5, RS6, RS7, RS8, RS9, RS10, RS11).

The management server 400 may calculate, from the pieces of relative pattern information P2, P3, P4, P5, P6, P7, P8, P9, P10, and P11, a psychological state value of an original author and relative psychological state values of imitators by using a correlation table.

In an index order based on an imitation order, the relative psychological state values may generate an imitation emotion pattern. The relative psychological state values of the imitators may be expressed as an increase or decrease such as −, +, or 0.

The device described above may be implemented as a hardware component, a software component, and/or a combination of the hardware component and the software component. For example, devices and components described in the embodiments may be implemented by using one or more general-purpose computers or special-purpose computers, like a processor, a controller, an arithmetic logic unit (ALU), a digital signal processor, a microcomputer, a field programmable gate array (FPGA), a programmable logic unit (PLU), a microprocessor, or any other device capable of executing and responding to instructions. A processing device may execute an operating system (OS) and one or more software applications executed by the OS. In addition, the processing device may also access, store, manipulate, process, and generate data in response to execution of software. For convenience of understanding, although a case in which one processing device is used is described, one of ordinary skill in the art will recognize that the processing device may include a plurality of processing elements and/or a plurality of types of processing elements. For example, the processing device may include a plurality of processors or one processor and one controller. In addition, other processing configurations are also possible, such as parallel processors.

Software may include a computer program, code, instructions, or a combination of one or more thereof, and may configure the processing device to operate as desired or, independently or collectively, instruct the processing device. Software and/or data may be permanently or temporarily embodied in any type of machine, component, physical device, virtual equipment, computer storage medium or device, or transmitted signal wave to be interpreted by a processing device or to provide instructions or data to the processing device. Software may be distributed over networked computer systems and stored or executed in a distributed manner. Software and data may be stored in one or more computer-readable recording media.

The method according to the embodiment may be implemented in the form of program instructions that may be executed through various computer units and recorded in a computer-readable recording medium. The computer-readable recording medium may include program instructions, data files, data structures, or the like, alone or in combination. The program instructions recorded on the medium may be specially designed and configured for the embodiment, or may be known and available to those skilled in the art of computer software. Examples of the computer-readable recording medium include hardware devices specially configured to store and execute program instructions such as a magnetic medium such as a hard disk, an optical medium such as a CD-ROM and a DVD, a magneto-optical medium such as a floptical disk, ROM, RAM, flash memory, or the like. Examples of program instructions include not only machine language code such as those generated by a compiler, but also high-level language code that may be executed by a computer using an interpreter or the like. The hardware devices described above may be configured to operate as one or more software modules to perform operations of the embodiments, and vice versa.

As described above, although the embodiments have been described with reference to the limited embodiments and drawings, various modifications and variations are possible from the above description by those skilled in the art. For example, even if the described techniques are performed in an order different from the described method, and/or the described components such as a system, a structure, a device, a circuit, or the like are combined or grouped in a different form from the described method, or replaced or substituted by other components or equivalents, appropriate results may be achieved.

According to an embodiment of the present disclosure, by generating pattern information obtained by digitally analyzing one or more pieces of drawing data of an imitation data group, the generated pattern information may be used to analyze psychological state values of users in the imitation data group.

Also, according to an embodiment of the present disclosure, a connection between the psychological state values of the users in the imitation group may be discriminated by generating relative pattern information between N pieces of drawing data, which are similar in shape and color.

In addition, according to an embodiment of the present disclosure, the relative pattern information may be analyzed by using a model trained by machine-training.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A method of generating relative pattern information between imitation drawing data, the method comprising:
collecting a first piece of drawing data generated by a first user and a second piece of drawing data generated by a second user and imitated from the first piece of drawing data;
generating a first imitation data group comprising the first piece of drawing data and the second piece of drawing data;
obtaining, from the first imitation data group, a first piece of pattern information and a second piece of pattern information corresponding to the first piece of drawing data and the second piece of drawing data, respectively;
calculating a first psychological state value and a second psychological state value corresponding the first piece of pattern information and the second piece of pattern information, respectively;
determining a relative psychological state value of the second psychological state value with respect to the first psychological state value;
calculating relative pattern information of the second piece of pattern information with respect to the first piece of pattern information; and
generating the relative psychological state value and the relative pattern information generated in the first imitation data group as a first imitation emotion pattern related to the first imitation data group.

2. The method of claim 1, further comprising:
receiving a third piece of drawing data generated by a third user; and
in response to the third piece of drawing data having a degree of similarity with the first or second piece of drawing data, the degree of similarity being greater than or equal to a preset minimum similarity value:
generating a third piece of pattern information corresponding to the third piece of drawing data by using the first imitation emotion pattern, and
determining a third psychological state value corresponding to the third piece of pattern information.

3. The method of claim 2, further comprising generating an imitation order map based on a generation time between the first piece of drawing data and the second piece of drawing data.

4. The method of claim 3, further comprising:
creating a community of one or more users who have drawn according to the first piece of drawing data as a group, the one or more users including at least one of the first user, the second user, and the third user; and
sharing drawing data of the one or more users in an online area of the community.

5. The method of claim 1, further comprising determining one of the first and second users as an original author and the other user as an imitator by using the relative psychological state value and the relative pattern information.

6. The method of claim 1, further comprising:
receiving a third piece of drawing data generated by a third user; and
in response to the third piece of drawing data having a degree of similarity with the first or second piece of drawing data, the degree of similarity being greater than or equal to a preset minimum similarity value:
calculating a third piece of pattern information corresponding to the third piece of drawing data, and
managing relative pattern information of the third piece of pattern information with respect to the first piece of pattern information by including the relative pattern information of the third piece of pattern information in the first imitation emotion pattern.

7. The method of claim 1, wherein the first and second pieces of drawing data each comprise at least one of a time value, a number of strokes, a stop time value, an erase time value, start coordinates, last coordinates, pen pressure, a velocity, a thickness, a color value, a red, blue, and green (RGB) ratio, a brightness value ratio, a hue ratio, and a color ratio.

8. A computer program stored in a computer-readable storage medium, wherein the computer program is executable by a processor, and wherein when the computer program is executed, the processor is configured to execute the method of claim 1.

9. A management server comprising:
a communication unit and a processor,
wherein the processor is configured to:
collect a first piece of drawing data generated by a first user and a second piece of drawing data generated by a second user and imitated from the first piece of drawing data;
generate a first imitation data group comprising the first piece of drawing data and the second piece of drawing data;
obtain, from the first imitation data group, a first piece of pattern information and a second piece of pattern information respectively corresponding to the first piece of drawing data and the second piece of drawing data, and calculate a first psychological state value and a second psychological state value respectively corresponding the first piece of pattern information and the second piece of pattern information;
determine a relative psychological state value of the second psychological state value with respect to the first psychological state value, and calculate relative pattern information of the second piece of pattern information with respect to the first piece of pattern information; and
generate the relative psychological state value and the relative pattern information generated in the first imitation data group as a first imitation emotion pattern related to the first imitation data group.

10. The management server of claim 9, wherein the processor is further configured to:
receive a third piece of drawing data generated by a third user; and,
in response to the third piece of drawing data having a degree of similarity with the first or second piece of drawing data, the degree of similarity being greater than or equal to a preset minimum similarity value, generate a third piece of pattern information corresponding to the third piece of drawing data by using the first imitation emotion pattern, and determine a third psychological state value corresponding to the third piece of pattern information.

11. The management server of claim 10, wherein the processor is further configured to generate an imitation order map based on a generation time between the first piece of drawing data and the second piece of drawing data.

12. The management server of claim 11, wherein the processor is further configured to:
create a community of one or more users who have drawn according to the first piece of drawing data as a group, the one or more users including at least one of the first user, the second user, and the third user; and
share drawing data of the one or more users in an online area of the community.

13. The management server of claim 9, wherein the processor is further configured to determine one of the first and second users as an original author and the other user as an imitator by using the relative psychological state value and the relative pattern information.

14. The management server of claim 9, wherein the processor is further configured to:
receive a third piece of drawing data generated by a third user; and
in response to the third piece of drawing data having a degree of similarity with the first or second piece of drawing data, the degree of similarity being greater than or equal to a preset minimum similarity value:
calculate a third piece of pattern information corresponding to the third piece of drawing data, and
manage relative pattern information of the third piece of pattern information with respect to the first piece of pattern information by including the relative pattern information of the third piece of pattern information in the first imitation emotion pattern.

15. The management server of claim 9, wherein the first and second pieces of drawing data each comprise at least one of a time value, a number of strokes, a stop time value, an erase time value, start coordinates, last coordinates, pen pressure, a velocity, a thickness, a color value, a red, blue, and green (RGB) ratio, a brightness value ratio, a hue ratio, and a color ratio.
